(19) United States
(12) Patent Application Publication
(10) Pub. No.: US 2024/0207381 A1
(43) Pub. Date: Jun. 27, 2024

(54) BIHAPTENIZED AUTOLOGOUS VACCINES AND USES THEREOF

(71) Applicant: BIOVAXYS, INC., New York, NY (US)

(72) Inventor: David Berd, Wyncote, PA (US)

(21) Appl. No.: 18/557,655

(22) PCT Filed: Apr. 27, 2022
(86) PCT No.: PCT/US2022/026461

§ 371 Date: Oct. 27, 2023

Related U.S. Application Data
(60) Provisional application No. 63/180,509, filed on Apr. 27, 2021.

(51) Int. Cl. A61K 39/00; C07K 16/28

(52) U.S. Cl. CPC A61K 39/0011; C07K 16/2818; A61K 2039/507; A61K 2039/5152; A61K 2039/545; A61K 2039/6012; A61K 2039/892

(57) ABSTRACT

In some embodiments. methods of treating cervical cancer, including cervical cancers that are resistant to immune checkpoint inhibitor therapy. and cervical cancers that do not respond to immune checkpoint inhibitor therapy or have acquired resistance to immune checkpoint inhibitor therapy are disclosed.

### FIELD OF THE INVENTION

The invention described herein relates generally to a method of treating cervical cancer using autologous vaccines in combination with other treatments.

### BACKGROUND OF THE INVENTION

Immunotherapy regimens using unmodified, intact tumor cells prepared from tumors taken from the patient have been extensively described in the literature (see, e.g. Berd et al. Cancer Research 1986; 46:2572-2577; Hoover et al. Cancer 1985; 55:1236-1243; and U.S. Pat. No. 5,484,596 to Hanna et al.). Alternative vaccine compositions based on disrupted cells have also been suggested including, for example, tumor cell membranes (see, e.g. Levin et al. Human Tumors in Short Term Culture: Techniques and Clinical Applications, P. P. Dendy, Ed., 1976, Academic Press, London, pp. 277-280) or tumor peptides extracted from tumors (see U.S. Pat. Nos. 5,550,214 and 5,487,556). The tumor cells can also be modified in some manner to alter or increase the immune response (see Hostetler et al. Cancer Research 1989; 49:1207-1213; and Muller et al. Anticancer Research 1991; 11:925-930).

One particular form of tumor cell modification that has a pronounced effect on immunotherapy is coupling of a hapten to the tumor cells. An autologous whole-cell vaccine modified with the hapten dinitrophenyl (DNP) has been shown to produce inflammatory responses in metastatic sites of melanoma patients. Adjuvant therapy with DNP-modified vaccine produces markedly higher post-surgical survival rates than those reported after surgery alone. U.S. Pat. No. 5,290,551 to Berd discloses and claims vaccine compositions comprising haptenized melanoma cells. Melanoma patients who were treated with these cells developed a strong immune response. This response can be detected in a delayed-type hypersensitivity (DTH) response to haptenized and non-haptenized tumor cells. More importantly, the immune response resulted in increased survival rates of melanoma patients.

Haptenized tumor cell vaccines have also been described for other types of cancers, including lung cancer, breast cancer, colon cancer, pancreatic cancer, ovarian cancer, and leukemia (see International Patent Publication No. WO 9640173 and WO 0009140, and U.S. Pat. No. 6,333,028, and the associated techniques and treatment regimens optimized (see International Patent Publication No. WO 0038710, WO 0031542, WO 9952546, and WO 9814206). For example, it has been shown that the addition of human serum albumin (HSA) increases the stability of haptenized tumor cell preparations (see WO 0029554 and U.S. Pat. No. 6,248,585).

It has also been determined that haptenization of tumor cell extracts, such as plasma membranes or peptides, can yield immunotherapy vaccines (see International Patent Publication No. WO 9640173 and WO 9940925).

Also developing in parallel has been therapeutic antibodies directed against checkpoint gene products (see Ribas, Science 359:1350-55 (2018). While these checkpoint therapies have been somewhat effective, there is still the problem of initial tumor insensitivity, acquired resistance, or a previously responsive tumor, becoming refractor to checkpoint therapy.

Treating these patients who have insensitive tumors, tumors that have acquired resistance, or become otherwise generally refractory to checkpoint therapy is a major technical problem in the field of cervical cancer treatment.

### SUMMARY OF THE INVENTION

Embodiments disclosed herein are methods of treating cervical cancer, the method comprising administering an autologous bihaptenized tumor vaccine alone or in combination with at least one checkpoint therapy. In some embodiments, the cervical cancer is metastatic cervical cancer. In some embodiments, the cervical cancer has acquired resistance to an immune checkpoint inhibitor. In some embodiments, the cervical cancer is insensitive to an immune checkpoint inhibitor.

### BRIEF DESCRIPTION OF THE DRAWINGS

The foregoing summary, as well as the following detailed description of the invention, will be better understood when read in conjunction with the appended drawings.

FIG. 1-A shows a typical positive, post-vaccine delayed type hypersensitivity response. FIG. 1-B shows a typical negative control (vehicle) and positive control (BCG) DTH response.

### DETAILED DESCRIPTION OF THE INVENTION

Unless defined otherwise, all technical and scientific terms used herein have the same

meaning as is commonly understood by one of skill in the art to which this invention belongs. All patents and publications referred to herein are incorporated by reference in their entireties.

In an embodiment, the invention provides a method of treating cervical cancer, the method comprising:
(i) administering an effective amount of an autologous bihaptenized vaccine, and optionally
(ii) administering an effective amount of at least one immune checkpoint inhibitor.

In some embodiments, the autologous bihaptenized vaccine is administered every other week for at least eight weeks. In some embodiments, the autologous bihaptenized vaccine is administered once per week for at least six weeks. In some embodiments, the method further comprising at least one booster injection of the autologous bihaptenized vaccine about six months after the first injection. In some embodiments, booster injections continue every six months until disease progression.

In an embodiment, the invention provides a method of treating cervical cancer, the method comprising: co-administering one or more compositions comprising therapeutically effective amounts of:
(i) at least one immune checkpoint inhibitor; and
(ii) an autologous bihaptenized vaccine.

In some embodiments, the effective amount of an autologous bihaptenized vaccine is administered every other week until the delayed type hypersensitivity diagnostic test is positive.

In some embodiments, the human subject is pre-cancerous for cervical cancer and is suffering from cervical intraepithelial neoplasia (CIN), squamous intraepithelial lesions (SIL), and dysplasia. In some embodiments, the human subject is suffering from CIN-1 (also called mild dysplasia or low grade SIL), where not much of the tissue looks abnormal, and it is considered the least serious cervical pre-cancer. In some embodiments the human subject is suffering from CIN-2 or CIN-3 (also known as moderate/severe dysplasia or high-grade SIL) where more of the tissue looks abnormal. In some embodiments the human subject is suffering from high-grade SIL, which is the most serious pre-cancer.

In some embodiments, the cervical cancer is squamous cell carcinoma or adenocarcinoma. Squamous cell carcinomas develop from cells in the exocervix. Squamous cell carcinomas most often begin in the transformation zone (where the exocervix joins the endocervix). Adenocarcinomas are cancers that develop from glandular cells. Cervical adenocarcinoma develops from the mucus-producing gland cells of the endocervix. In some embodiments, cervical cancers have features of both squamous cell carcinomas and adenocarcinomas. These are called adenosquamous carcinomas or mixed carcinomas.

In some embodiments, the at least one immune checkpoint inhibitor is selected from the group consisting of CTLA-4, PD-1, PD-L1, LAG3, B7-H3, B7-H4, KIR, OX40, IDO-1, IDO-2, CEACAM1, INFR5F4, BTLA, OX4OL, and TIM3 or combinations thereof. In some preferred embodiments, the immune checkpoint inhibitor is PD-1. In some embodiments, the PD-1 immune checkpoint inhibitor is selected from the group consisting of nivolumab, pembrolizumab, atezolizumab, avelumab, and durvalumab.

In some embodiments, the immune checkpoint inhibitor is an antibody to CTLA-4. In some embodiments, the CTLA-4 immune checkpoint inhibitor is selected from the group consisting of ipilimumab and tremelimumab.

In some embodiments, the at least one immune checkpoint inhibitor comprises a PD-1 immune checkpoint inhibitor and a CTLA-4 immune checkpoint inhibitor.

In an embodiment, the invention provides a personalized diagnostic test kit for cervical cancer, the kit comprising:
(i) one or more single dose filled vials wherein the vial contains bihaptenized autologous tumor cells in a pharmaceutically acceptable carrier;
(ii) one or more single dose vials wherein the vial contains a test negative control in a pharmaceutically acceptable carrier;
(iii) written instructions; and
(iv) a guide for scoring the test results.

In an embodiment, the invention provides a method of treating cervical cancer, the method comprising:
(i) administering an effective amount of at least one immune checkpoint inhibitor; and,
(ii) administering an effective amount of an autologous bihaptenized vaccine wherein the autologous bihaptenized vaccine is prepared by a process comprising the steps of:
   (a) washing a dissected cervical tumor fragment, wherein the fragment is at least about one cm in diameter;
   (b) dissociating the cervical tumor fragment into a suspension of cells;
   (c) irradiating the suspension of cells with gamma radiation;
   (d) dividing the irradiated suspension of cells into at least two approximately equal portions;
   (e) haptenizing a first portion with a first haptenization reagent and a second portion with second haptenization reagent;
   (f) fixing each portion;
   (g) combining the portions; and,
   (h) aliquoting cells comprising the product of step (g) into aliquots of about 6×10⁶ to about 50×10⁶ cell/mL.

In one embodiment, the dose of the gamma radiation is about 1000, 1500, 2000, 2500, 3000, 3500, 4000, 4500, or 5000 cGy. In one embodiment, each portion is fixed with about 35%, 37.5%, 40%, 42.5%, 45%, 47.5%, or 50% ethanol.

### Definitions

The following definitions are used in connection with the invention disclosed herein. Unless defined otherwise, all technical and scientific terms used herein have the same meaning as commonly understood to one of skill in the art to which this invention belongs.

The term “autologous” means that a patient's own cells are used to prepare the embodiments disclosed herein.

The term “immune checkpoint inhibitor” means a therapeutic that reduces or decreases the cellular function of an immune checkpoint gene or gene product. Any suitable immune checkpoint inhibitor is contemplated for use with the methods disclosed herein “immune checkpoint inhibitors,” as used herein refer to any modulator that inhibits the activity of the immune checkpoint molecule. Checkpoint inhibitors can include, but are not limited to, immune checkpoint molecule binding proteins, small molecule inhibitors, antibodies, antibody-derivatives (including Fab fragments and scFvs), antibody-drug conjugates, antisense oligonucleotides, siRNA, aptamers, peptides and peptide mimetics. Inhibitory nucleic acids that decrease the expression and/or activity of immune checkpoint molecules can also be used in the methods disclosed herein. Herein, the term “immune checkpoint inhibitor” and “checkpoint inhibitor” are used interchangeably.

The terms “co-administration” or “co-administering” or “administered in combination with” or “administering in combination with” or “simultaneous” or “concurrent” as used herein, encompass administration of two or more active pharmaceutical ingredients to a subject so that both active pharmaceutical ingredients and/or their metabolites are present in the subject at the same time. Co-administration includes simultaneous administration in separate compositions, administration at different times in separate compositions, or administration in a composition in which two or more active pharmaceutical ingredients are present. Administration at different times in separate compositions is preferred.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g. the weight, age and gender of the subject), the severity of the disease condition, the manner of administration etc. which can readily be determined by one of ordinary skill in the art. The term also applies to a dose that will induce a particular response in target cells (e.g. the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

A “therapeutic effect” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

“Pharmaceutically acceptable carrier” or “pharmaceutically acceptable excipient” is intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the invention is contemplated. Additional active pharmaceutical ingredients, such as other drugs, can also be incorporated into the described compositions and methods.

When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, preferably from 0% to 10%, more preferably from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

The terms “antibody” and its plural form “antibodies” refer to whole immunoglobulins and any antigen-binding fragment (“antigen-binding portion”) or single chains thereof. An “antibody” further refers to a glycoprotein comprising at least two heavy (H) chains and two light (L) chains inter-connected by disulfide bonds, or an antigen-binding portion thereof. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, C_{L}. The V_{H} and V_{L} regions of an antibody may be further subdivided into regions of hypervariability, which are referred to as complementarity determining regions (CDR) or hypervariable regions (HVR), and which can be interspersed with regions that are more conserved, termed framework regions (FR). Each V_{H} and V_{L} is composed of three CDRs and four FRS, arranged from amino-terminus to carboxy-terminus in the following order: FR1, CDR1, FR2, CDR2, FR3, CDR3, FR4. The variable regions of the heavy and light chains contain a binding domain that interacts with an antigen epitope or epitopes. The constant regions of the antibodies may mediate the binding of the immunoglobulin to host tissues or factors, including various cells of the immune system (e.g. effector cells) and the first component (Clq) of the classical complement system.

The terms “monoclonal antibody” (mAb), “monoclonal antibody composition” or their plural forms refer to a preparation of antibody molecules of single molecular composition. A monoclonal antibody composition displays a single binding specificity and affinity for a particular epitope. Monoclonal antibodies specific to, e.g. CTLA-4, PD-1, PD-L1, LAG3, B7-H3, B7-H4, KIR, OX40, IDO-1, IDO-2, CEACAM1, INFR5F4, BTLA, OX4OL, or TIM3 can be made using knowledge and skill in the art of injecting test subjects with CTLA-4, PD-1, PD-L1, LAG3, B7-H3, B7-H4, KIR, OX40, IDO-1, IDO-2, CEACAM1, INFR5F4, BTLA, OX4OL, or TIM3 antigen and then isolating hybridomas expressing antibodies having the desired sequence or functional characteristics. DNA encoding the monoclonal antibodies is readily isolated and sequenced using conventional procedures (e.g. by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such asE. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies will be described in more detail below.

The terms “antigen-binding portion” or “antigen-binding fragment” of an antibody (or simply “antibody portion”) as used herein, refers to one or more fragments of an antibody that retain the ability to specifically bind to an antigen (e.g. CTLA-4, PD-1, PD-L1, LAG3, B7-H3, B7-H4, KIR, OX40, IDO-1, IDO-2, CEACAM1, INFR5F4, BTLA, OX4OL, or TIM3). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and CH1 domains; (ii) a F(ab')2 fragment, a bivalent fragment comprising two Fab fragments linked by a disulfide bridge at the hinge region; (iii) a Fd fragment consisting of the V_{H} and CH1 domains; (iv) a Fv fragment consisting of the Vi and VH domains of a single arm of an antibody, (v) a domain antibody (dAb) fragment (Ward et al. Nature, 1989, 341, 544-546), which may consist of a V_{H} or a V_{L} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv); see, e.g. Bird et al. Science 1988, 242, 423-426; and Huston et al. Proc. Natl. Acad. Sci. USA 1988, 85, 5879-5883). Such scFv antibodies are also intended to be encompassed within the terms “antigen-binding portion” or “antigen-binding fragment” of an antibody. These antibody fragments are obtained using conventional techniques known to those with skill in the art, and the fragments are screened for utility in the same manner as are intact antibodies.

The term “human antibody” as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g. mutations introduced by random or site-specific mutagenesis in vitro or by somatic mutation in vivo). The term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, e.g. a transgenic mouse, having a genome comprising a human heavy chain transgene and a light chain transgene fused to an immortalized cell.

The term “recombinant human antibody” as used herein, includes all human antibodies that are prepared, expressed, created or isolated by recombinant means, such as (a) antibodies isolated from an animal (e.g. a mouse) that is transgenic or transchromosomal for human immunoglobulin genes or a hybridoma prepared therefrom (described further below), (b) antibodies isolated from a host cell transformed to express the human antibody, e.g. from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human lg sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g. a conjugate of the antibody and another active pharmaceutical ingredient or antibody. The terms “conjugate,” “antibody-drug conjugate”, “ADC,” or “immunoconjugate” refers to an antibody, or a fragment thereof, conjugated to a therapeutic moiety, such as a bacterial toxin, a cytotoxic drug or a radionuclide-containing toxin. Toxic moieties can be conjugated to antibodies of the invention using methods available in the art.

The terms “humanized antibody” or “humanized antibodies” or “humanized” are intended to refer to antibodies in which CDR sequences derived from the germline of another mammalian species, such as a mouse, have been grafted onto human framework sequences. Additional framework region modifications may be made within the human framework sequences. Humanized forms of non-human (for example, murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a 15 hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al. Nature 1986, 321, 522-525; Riechmann et al. Nature 1988, 332, 323-329; and Presta, Curr. Op. Struct. Biol. 1992, 2, 593-596.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

A “diabody” is a small antibody fragment with two antigen-binding sites. The fragments comprises a heavy chain variable domain (VH) connected to a light chain variable domain (Vi) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully in, e.g. European Patent No. EP 404,097, International Patent Publication No. WO 93/11161; and Bolliger et al. Proc. Natl. Acad. Sci. USA 1993, 90, 6444-6448. Diabody, as used herein, also encompasses bi-specific antibodies.

The term “glycosylation” refers to a modified derivative of an antibody. An aglycoslated antibody lacks glycosylation. Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. A glycosylation may increase the affinity of the antibody for antigen, as described in U.S. Pat. Nos. 5,714,350 and 6,350,861. Additionally, or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, the cell lines Ms704, Ms705, and Ms709 lack the fucosyltransferase gene, FUT8 (α-(1,6) fucosyltransferase), such that antibodies expressed in the Ms704, Ms705, and Ms709 cell lines lack fucose on their carbohydrates. The Ms704, Ms705, and Ms709 FUT8-/-cell lines were created by the targeted disruption of the FUT8 gene in CHO/DG44 cells using two replacement vectors (see e.g. U.S. Patent Publication No. 2004/0110704 or Yamane-Ohnuki et al. Biotechnol. Bioeng., 2004, 87, 614-622). As another example, European Patent No. EP 1176195 describes a cell line with a functionally disrupted FUT8 gene, which encodes a fucosyl transferase, such that antibodies expressed in such a cell line exhibit hypofucosylation by reducing or eliminating the a-1,6 bond-related enzyme, and also describes cell lines which have a low enzyme activity for adding fucose to the N-acetylglucosamine that binds to the Fc region of the antibody or does not have the enzyme activity, for example the rat myeloma cell line YB2/0 (ATCC CRL 1662). International Patent Publication WO 03/035835 describes a variant CHO cell line, Lec 13 cells, with reduced ability to attach fucose to Asn(297)-linked carbohydrates, also resulting in hypofucosylation of antibodies expressed in that host cell (see also Shields et al. J. Biol. Chem. 2002, 277, 26733-26740. International Patent Publication WO 99/54342 describes cell lines engineered to express glycoprotein-modifying glycosyl transferases (e.g. β-(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al. Nat. Biotech. 1999, 17, 176-180). Alternatively, the fucose residues of the antibody may be cleaved off using a fucosidase enzyme. For example, the fucosidase a-L-fucosidase removes fucosyl residues from antibodies as described in Tarentino et al. Biochem. 1975, 14, 5516-5523.

“Pegylation” refers to a modified antibody, or a fragment thereof, that typically is reacted with polyethylene glycol (PEG), such as a reactive ester or aldehyde derivative of PEG, under conditions in which one or more PEG groups become attached to the antibody or antibody fragment. Pegylation may, for example, increase the biological (e.g. serum) half-life of the antibody. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C₁-C₁₀) alkoxy- or aryloxy-polyethylene glycol or polyethylene glycol-maleimide. The antibody to be pegylated may be an aglycosylated antibody. Methods for pegylation are known in the art and can be applied to the antibodies of the invention, as described for example in European Patent No. EP 0154316 and EP 0401384.

The term “biosimilar” means a biological product that is highly similar to a U.S. licensed reference biological product notwithstanding minor differences in clinically inactive components, and for which there are no clinically meaningful differences between the biological product and the reference product in terms of the safety, purity, and potency of the product. Furthermore, a similar biological or “biosimilar” medicine is a biological medicine that is similar to another biological medicine that has already been authorized for use by the European Medicines Agency. The term “biosimilar” is also used synonymously by other national and regional regulatory agencies. Biological products or biological medicines are medicines that are made by or derived from a biological source, such as a bacterium or yeast. They can consist of relatively small molecules such as human insulin or erythropoietin, or complex molecules such as monoclonal antibodies. For example, if the reference anti-CD20 monoclonal antibody is rituximab, an anti-CD20 biosimilar monoclonal antibody approved by drug regulatory authorities with reference to rituximab is a “biosimilar to” rituximab or is a “biosimilar thereof” of rituximab. In Europe, a similar biological or “biosimilar” medicine is a biological medicine that is similar to another biological medicine that has already been authorized for use by the European Medicines Agency (EMA). The relevant legal basis for similar biological applications in Europe is Article 6 of Regulation (EC) No 726/2004 and Article 10(4) of Directive 2001/83/EC, as amended and therefore in Europe, the biosimilar may be authorized, approved for authorization or subject of an application for authorization under Article 6 of Regulation (EC) No 726/2004 and Article 10(4) of Directive 2001/83/EC. The already authorized original biological medicinal product may be referred to as a “reference medicinal product” in Europe. Some of the requirements for a product to be considered a biosimilar are outlined in the CHMP Guideline on Similar Biological Medicinal Products. In addition, product specific guidelines, including guidelines relating to monoclonal antibody biosimilars, are provided on a product-by-product basis by the EMA and published on its website. A biosimilar as described herein may be similar to the reference medicinal product by way of quality characteristics, biological activity, mechanism of action, safety profiles and/or efficacy. In addition, the biosimilar may be used or be intended for use to treat the same conditions as the reference medicinal product. Thus, a biosimilar as described herein may be deemed to have similar or highly similar quality characteristics to a reference medicinal product. Alternatively, or in addition, a biosimilar as described herein may be deemed to have similar or highly similar biological activity to a reference medicinal product. Alternatively, or in addition, a biosimilar as described herein may be deemed to have a similar or highly similar safety profile to a reference medicinal product. Alternatively, or in addition, a biosimilar as described herein may be deemed to have similar or highly similar efficacy to a reference medicinal product. As described herein, a biosimilar in Europe is compared to a reference medicinal product which has been authorized by the EMA. However, in some instances, the biosimilar may be compared to a biological medicinal product which has been authorized outside the European Economic Area (a non-EEA authorized “comparator”) in certain studies. Such studies include for example certain clinical and in vivo non-clinical studies. As used herein, the term “biosimilar” also relates to a biological medicinal product which has been or may be compared to a non-EEA authorized comparator. Certain biosimilars are proteins such as antibodies, antibody fragments (for example, antigen binding portions) and fusion proteins. A protein biosimilar may have an amino acid sequence that has minor modifications in the amino acid structure (including for example deletions, additions, and/or substitutions of amino acids) which do not significantly affect the function of the polypeptide. The biosimilar may comprise an amino acid sequence having a sequence identity of 97% or greater to the amino acid sequence of its reference medicinal product, e.g. 97%, 98%, 99% or 100%. The biosimilar may comprise one or more post-translational modifications, for example, although not limited to, glycosylation, oxidation, deamidation, and/or truncation which is/are different to the post-translational modifications of the reference medicinal product, provided that the differences do not result in a change in safety and/or efficacy of the medicinal product. The biosimilar may have an identical or different glycosylation pattern to the reference medicinal product. Particularly, although not exclusively, the biosimilar may have a different glycosylation pattern if the differences address or are intended to address safety concerns associated with the reference medicinal product. Additionally, the biosimilar may deviate from the reference medicinal product in for example its strength, pharmaceutical form, formulation, excipients and/or presentation, providing safety and efficacy of the medicinal product is not compromised. The biosimilar may comprise differences in for example pharmacokinetic (PK) and/or pharmacodynamic (PD) profiles as compared to the reference medicinal product but is still deemed sufficiently similar to the reference medicinal product as to be authorized or considered suitable for authorization. In certain circumstances, the biosimilar exhibits different binding characteristics as compared to the reference medicinal product, wherein the different binding characteristics are considered by a Regulatory Authority such as the EMA not to be a barrier for authorization as a similar biological product. The term “biosimilar” is also used synonymously by other national and regional regulatory agencies.

The term “solid tumor” refers to an abnormal mass of tissue that usually does not contain cysts or liquid areas. Solid tumors may be benign or malignant. The term “solid tumor cancer” refers to malignant, neoplastic, or cancerous solid tumors. Solid tumor cancers include, but are not limited to, sarcomas, carcinomas, and lymphomas, such as cancers of the cervix. The tissue structure of solid tumors includes interdependent tissue compartments including the parenchyma (cancer cells) and the supporting stromal cells in which the cancer cells are dispersed and which may provide a supporting microenvironment.

The term “microenvironment” as used herein, may refer to the tumor microenvironment as a whole or to an individual subset of cells within the microenvironment.

The terms “sequence identity” or “percent identity” or “sequence percent identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences. Suitable programs to determine percent sequence identity include for example the BLAST suite of programs available from the U.S. Government's National Center for Biotechnology Information BLAST web site. Comparisons between two sequences can be carried using either the BLASTN or BLASTP algorithm. BLASTN is used to compare nucleic acid sequences, while BLASTP is used to compare amino acid sequences. ALIGN, ALIGN-2 (Genentech, South San Francisco, California) or MegAlign, available from DNASTAR, are additional publicly available software programs that can be used to align sequences. One skilled in the art can determine appropriate parameters for maximal alignment by particular alignment software. In certain embodiments, the default parameters of the alignment software are used.

Certain embodiments of the present invention comprise a variant of an antibody, e.g. an anti-CTLA-4 or anti-LAG3 antibody and/or an anti-IDO-1 antibody and/or an anti-PD-1 antibody, anti-PD-L1 and/or an anti-PD-L2 antibody. As used herein, the term “variant” encompasses but is not limited to antibodies which comprise an amino acid sequence which differs from the amino acid sequence of a reference antibody by way of one or more substitutions, deletions and/or additions at certain positions within or adjacent to the amino acid sequence of the reference antibody. The variant may comprise one or more conservative substitutions in its amino acid sequence as compared to the amino acid sequence of a reference antibody. Conservative substitutions may involve, e.g. the substitution of similarly charged or uncharged amino acids. The variant retains the ability to specifically bind to the antigen of the reference antibody.

For the avoidance of doubt, it is intended herein that particular features (for example integers, characteristics, values, uses, diseases, formulae, compounds or groups) described in conjunction with a particular aspect, embodiment or example of the invention are to be understood as applicable to any other aspect, embodiment or example described herein unless incompatible therewith. Thus such features may be used where appropriate in conjunction with any of the definition, claims or embodiments defined herein. All of the features disclosed in this specification (including any accompanying claims, abstract and drawings), and/or all of the steps of any method or process so disclosed, may be combined in any combination, except combinations where at least some of the features and/or steps are mutually exclusive. The invention is not restricted to any details of any disclosed embodiments. The invention extends to any novel one, or novel combination, of the features disclosed in this specification (including any accompanying claims, abstract and drawings), or to any novel one, or any novel combination, of the steps of any method or process so disclosed.

### Methods of Preparing Autologous Vaccines

The preparation of autologous vaccines using a single hapten reagent are known to the skilled artisan. For example, U.S. Pat. No. 5,290,551 discloses the preparation of autologous vaccines from patient melanoma tumor samples using a single hapten reagent. U.S. Pat. No. 5,290,551 is incorporated by reference in its entirety. Methods of preserving tumor samples are discussed by Berd in

U.S. Published Patent Application No. 20030170756, which is incorporated by reference, particularly for methods of preserving tumor cells.

In an embodiment, the invention provides for an autologous personalized bihaptenized vaccine, such a tumor vaccine may be prepared as follows: autologous bihaptenized vaccine is prepared by a process comprising the steps of:
(a) washing a dissected cervical tumor fragment, wherein the fragment is at least about one cm in diameter;
(b) dissociating the cervical tumor fragment into a suspension of cells;
(c) irradiating the suspension of cells with gamma radiation;
(d) dividing the irradiated suspension of cells into at least two approximately equal portions;
(e) haptenizing a first portion with a first haptenization reagent and a second portion with second haptenization reagent;

(f) fixing each portion;

(g) combining the portions; and, (h) aliquoting cells comprising the product of step (g) into aliquots of about 6×10⁶ to about 50×10⁶ cell/mL.

In one embodiment, the dose of the gamma radiation is about 1000, 1500, 2000, 2500, 3000, 3500, 4000, 4500, or 5000 cGy. In one embodiment, each portion is fixed with about 35%, 37.5%, 40%, 42.5%, 45%, 47.5%, or 50% ethanol.

The step of haptenizing may be performed by modifying a first aliquot of cells with DNP by a 30-minute incubation with the first haptenization reagent 2, 4-difluoronitrobenzene (DNFB), and then modifying a second aliquot of cells with the second haptenization reagent sulfanilic acid (SA) by a 5-minute incubation with the diazonium salt of SA. The haptenized the cells are washed with HBSS, counted, and fixed with ethanol at a final concentration of about 37.5% for about 10 minutes. A second haptenization reagent may be selected from those known in the art, for example, those described in Nahas, Cell. Immunol. 54:241-247 (1980). Preferred haptenization reagents may target the ε-amino group of amino acid.

The dissociation of the tumor fragment into a suspension of cells may be achieved by methods known in the art. In preferred embodiments, the tumor fragment is dissociated into a suspension of cells by a means for mechanical dissociation.

In some embodiments, the first haptenization reagent and the second haptenization reagent each independently is selected from trinitrochlorobenzene (TNCB), 2,4-difluoronitrobenzene (DNFB), N-iodoacetyl-N′-(5-sulfonic-1-naphthyl)ethylenediamine (AED), sulfanilic acid (SA), trinitrophenol (TNP), 2,4,6-trinitrobenzenesulfonic acid (TNBS) and combinations thereof. In one embodiment, the first haptenization reagent is different from the second haptenization reagent.

In some embodiments, a bihaptenized vaccine dose is in the range between about 4×106 cells and about 50×106 cells. In some embodiments, a bihaptenized vaccine dose is about 8×10⁶ cells; about 9×10⁶ cells; about 10×10⁶ cells; about 11×10⁶ cells; about 12×10⁶ cells; about 13×10⁶ cells; about 14×10⁶ cells; about 15×10⁶ cells; about 16×10⁶ cells; about 17×10⁶ cells; about 18×10⁶ cells; about 19×10⁶ cells; about 20×10⁶ cells; about 21×10⁶ cells; 22×10⁶ cells; about 23×10⁶ cells; about 24 x 10⁶ cells; about 25×10⁶ cells; about 26×10⁶ cells; about 27×10⁶ cells; about 28×10⁶ cells; about 29 x 10⁶ cells; or about 30×10⁶ cells. In some embodiments, a bihaptenized vaccine dose is about 12 × 106 cells.

### Cryopreservation of Vaccines

Methods for cryopreserving haptenized cells are known to the skilled artisan. For example, Berd et al. disclose approaches for osmotic protection and controlled temperature freezing of haptenized cells in U.S. Pat. No. 8,435,784, which is incorporated by reference in its entirety.

### Co-administration of Compounds

Co-administration of compounds is a valuable clinical approach, particularly in the for the emerging immune checkpoint inhibitor agents. Predicting, before treatment, which patients may benefit from a particular immune checkpoint inhibitor remains unclear. E.g. Snyder et al. Genetic Basis for Clinical Response to CTLA-4 Blockade in Melanoma, N. Engl. J. of Med. 371:2189-2199 (2014).

An aspect of the invention is a composition, such as a pharmaceutical composition, comprising a combination of an immune checkpoint inhibitor and an autologous bihaptenized tumor vaccine. A method of treating cancer comprising: (i) administering an effective amount of at least one immune checkpoint inhibitor; and (ii) administering an effective amount of an autologous bihaptenized vaccine. In an embodiment, the autologous bihaptenized vaccine is administered every other week for at least eight weeks. In an embodiment, the autologous bihaptenized vaccine is administered every week for at least seven weeks. In an embodiment, the autologous bihaptenized vaccine is administered with or without adjuvant. In an embodiment, the adjuvant is Bacille Calmette-Guerin. In an embodiment, the adjuvant is selected from the group consisting of bacterial lipopolysaccharides, bacterial lipoproteins, antimicrobial peptides, saponins, lipoteichoic acid, squalene, immunostimulatory oligonucleotides, single-stranded RNA, synthetic phospholipids, MF59, E6020, IC31, lipopeptides, imidazoquinoline compounds, benzonaphthyridine compounds, and combinations thereof.

In some embodiments, the at least one immune checkpoint inhibitor is selected from the group consisting of CTLA-4, PD-1, PD-L1, LAG3, B7-H3, B7-H4, KIR, OX40, IDO-1, IDO-2, CEACAM1, INFR5F4, BTLA, OX4OL, and TIM3 or combinations thereof.

In an embodiment, the at least one immune checkpoint inhibitor is an inhibitor of the PD-1 immune checkpoint. In an embodiment, the at least one immune checkpoint inhibitor is an inhibitor of the CTLA-4 immune checkpoint.

### Immune Checkpoint Inhibitors

The invention encompasses a method for treating cervical cancer in a human female, comprising administering an effective amount of at least one immune checkpoint inhibitor, wherein the at least one immune checkpoint inhibitor is selected from the group consisting of CTLA-4, PD-1, PD-L1, LAG3, B7-H3, B7-H4, KIR, OX40, IDO-1, IDO-2, CEACAM1, INFR5F4, BTLA, OX4OL, and TIM3 or combinations thereof.

Moore provides concise summary of the FDA-approved immune checkpoint inhibitors in Immunotherapy in Cancer Treatment: A Review of Checkpoint Inhibitors, U.S. Pharm. 43(2): 27-31 (2018). The compositions, methods, and processes of the invention are understood to be functional in combination with inhibitors of immune checkpoints.

### PD-L1 Inhibitors

In some embodiments, the immune checkpoint inhibitor is an inhibitor of PD-LI. In some embodiments, the immune checkpoint inhibitor is an antibody against PD-1. In some embodiments, the immune checkpoint inhibitor is a monoclonal antibody against PD-1. In other or additional embodiments, the immune checkpoint inhibitor is a human or humanized antibody against PD-1. For example, the inhibitors of PD-1 biological activity (or its ligands) disclosed in U.S. Pat. No. 7,029,674; 6,808,710; or U.S. Pulbished Patent Application No. 20050250106 and 20050159351 can be used in the methods provided herein. Exemplary antibodies against PD-1 include: Anti-mouse PD-1 antibody Clone J43 (Bio X Cell); Anti-mouse PD-1 antibody Clone RMP1-14 (Bio X Cell); mouse anti-PD-1 antibody Clone EH12; Merck's MK-3475 anti-mouse PD-1 antibody (Keytruda™, pembrolizumab, lambrolizumab); and anti-PD-1 antibody, known as ANB011 (AnaptysBio); antibody MDX-1 106 (ONO-4538); human lgG4 monoclonal antibody nivolumab (Opdivo™, BMS-936558, MDX1106); AMP-514 and AMP-224 (AstraZeneca); and Pidilizumab (CT-011) by CureTech Ltd. Additional exemplary anti-PD-1 antibodies and methods for their use are described by Goldberg, Blood 110:186-192 (2007), Thompson, Clin. Cancer Res. 13(6): 1757-1761 (2007), and International Application No.WO 2006121168), each of which are expressly incorporated by reference herein. In some embodiments, the anti-PD-1 antibody is an anti-PD-1 antibody disclosed in any of the following patent publications (herein incorporated by reference): WO014557; WO 2011110604; WO 2008156712; US2012023752; WO 2011110621; WO 2004072286; WO 2004056875; WO 20100036959; WO 2010029434; WO 2016057898; W0201213548; WO 2002078731; WO 2012145493; WO 2010089411; WO 2001014557; WO 2013022091; WO 2013019906; WO 2003011911; and WO 2010001617.

In some embodiments, the PD-1 inhibitor is a PD-1 binding protein as disclosed in WO 200914335 (incorporated herein by reference).

In some embodiments, the PD-1 inhibitor is a peptidomimetic inhibitor of PD- 1 as disclosed in WO 2013132317 (incorporated herein by reference).

In some embodiments, the PD-1 inhibitor is an anti-mouse PD-1 mAb: clone J43 (Bio X Cell). In some embodiments, the immune checkpoint inhibitor is the anti-PD-1 antibody therapeutic cemiplimam-rwlc (Libtayo), jointly marketed by Regneron and Sanofi.

In some embodiments, the PD-1 inhibitor is a PD-L1 protein, a PD-L2 protein, or fragments, as well as antibody MDX-1 106 (ONO-4538) tested in clinical studies for the treatment of certain malignancies (Brahmer, J. Clin. Oncol. 28(19): 3167-75 (2010)). Other blocking antibodies may be readily identified and prepared by the skilled person based on the known domain of interaction between PD-1 and PD-L1/PD-L2, as described above.

### CTLA-4 Inhibitors

In some embodiments, the at least one immune checkpoint inhibitor is an inhibitor of CTLA-4. In some embodiments, the at least one immune checkpoint inhibitor is an antibody against CTLA-4. In some embodiments, the at least one immune checkpoint inhibitor is a monoclonal antibody against CTLA-4. In other or additional embodiments, the at least one immune checkpoint inhibitor is a human or humanized antibody against CTLA-4. In one embodiment, the anti-CTLA-4 antibody blocks the binding of CTLA-4 to CD80 (B7-1) and/or CD86 (B7-2) expressed on antigen presenting cells. Exemplary antibodies against CTLA-4 include: anti-CTLA-4 antibody ipilimumab (also known as Yervoy™, MDX-010, BMS-734016 and MDX-101); anti-CTLA4 Antibody, clone 9H10 from Millipore; tremelimumab (CP-675,206, ticilimumab); and anti-CTLA4 antibody clone BNI3 from Abcam.

In some embodiments, the anti-CTLA-4 antibody is an anti-CTLA-4 antibody disclosed in any of the following patent publications (which is incorporated by reference in its entirety): WO 2001014424; WO 2004035607; EP 1212422; WO 2003086459; WO 2012120125; WO 2000037504; WO 2009100140; WO 200609649; WO 2005092380; WO 2007123737; WO 2006029219; WO 20100979597; WO 200612168; and WO 1997020574. Additional CTLA-4 antibodies are described in U.S. Patent No. 5,811,097, 5,855,887, 6,051,227, and 6,984,720; in International Patent Publication No. WO 0114424 and WO 0037504; and in U.S. Patent Application Publication No. 20020039581 and 2002086014; and/or U.S. Pat. No. 5,977,318, 6,682,736, 7, 109,003, and 7,132,281, incorporated herein by reference.

In some embodiments, the anti-CTLA-4 antibody is an, for example, those disclosed in: WO 98/42752; U.S. Pat. No. 6,682,736 and 6,207,156; Hurwitz, Proc. Natl. Acad. Sci. USA, 95(17): 10067-10071 (1998); Camacho, J. Clin. Oncol., 22(145): Abstract No. 2505 (2004) (antibody CP-675206); Mokyr, Cancer Res., 58:5301-5304 (1998).

In some embodiments, the CTLA-4 inhibitor is a CTLA-4 ligand as disclosed in WO 1996040915. In other embodiments the CTLA-4 inhibitor may be B7-like peptides or nucleic acid molecules disclosed in U.S. Pat. No. 6,630,575.

### Methods of Treatment of Cervical Cancer

In some embodiments, the methods of treatment of the present invention are for use in the treatment of cervical cancer. In some embodiments, the cervical cancer is squamous cell carcinoma or adenocarcinoma. Squamous cell carcinomas develop from cells in the exocervix. Squamous cell carcinomas most often begin in the transformation zone (where the exocervix joins the endocervix). Adenocarcinomas are cancers that develop from glandular cells. Cervical adenocarcinoma develops from the mucus-producing gland cells of the endocervix. In some embodiments, cervical cancers have features of both squamous cell carcinomas and adenocarcinomas. These are called adenosquamous carcinomas or mixed carcinomas.

In some embodiments, the methods of treatment encompass treatment of a human subject is pre-cancerous for cervical cancer and is suffering from cervical intraepithelial neoplasia (CIN), squamous intraepithelial lesions (SIL), and dysplasia. In some embodiments, the human subject is suffering from CIN-1 (also called mild dysplasia or low grade SIL), and is where not much of the tissue looks abnormal, and it is considered the least serious cervical pre-cancer. In some embodiments the human subject is suffering from CIN-2 or CIN-3 (also known as moderate/severe dysplasia or high-grade SIL) where more of the tissue looks abnormal. In some embodiments the human subject is suffering from high-grade SIL, which is the most serious pre-cancer.

An embodiment of the invention provides a method of treating cervical cancer, the method comprising: (i) administering an effective amount of at least one immune checkpoint inhibitor; and, (ii) administering an effective amount of an autologous bihaptenized vaccine. In some embodiments, steps (i) and steps (ii) occur within the same time period. In some embodiments, the autologous bihaptenized vaccine is administered every other week for at least eight weeks. In some embodiments, the autologous bihaptenized vaccine is administered once per week for at least six weeks.

In an embodiment, the invention provides a method of treating cervical cancer, the method comprising: co-administering one or more compositions comprising therapeutically effective amounts of: (i) at least one immune checkpoint inhibitor; and, (ii) an autologous bihaptenized vaccine.

In some embodiments, the method further comprising at least one booster injection of the autologous bihaptenized vaccine to a human subject about six months after the first injection. In some embodiments, the effective amount of an autologous bihaptenized vaccine is administered every other week until the delayed type hypersensitivity diagnostic test is positive.

In some embodiments, the invention provides a method of treating metastatic cervical cancer. In some embodiments, the cancer has acquired resistance to a checkpoint inhibitor.

An embodiment of the invention provides a method of treating cervical cancer, the method comprising: (i) administering an effective amount of at least one immune checkpoint inhibitor; and, (ii) administering an effective amount of an autologous bihaptenized vaccine, wherein the at least one immune checkpoint inhibitor is selected from the group consisting of CTLA-4, PD-1, PD-L1, LAG3, B7-H3, B7-H4, KIR, OX40, IDO-1, IDO-2, CEACAM1, INFR5F4, BTLA, OX4OL, and TIM3 or combinations thereof. In some embodiments, the at least one immune checkpoint inhibitor comprises PD-1. In some embodiments, the PD-1 immune checkpoint inhibitor is selected from the group consisting of nivolumab, pembrolizumab, atezolizumab, avelumab, and durvalumab.

In some embodiments, the at least one immune checkpoint inhibitor comprises CTLA-4. In some embodiments, the CTLA-4 immune checkpoint inhibitor is selected from the group consisting of ipilimumab and tremelimumab.

In some embodiments, the at least one checkpoint inhibitor comprises a PD-1 immune checkpoint inhibitor and a CTLA-4 immune checkpoint inhibitor.

In some embodiments, the cervical cancer is resistant to immune checkpoint inhibitor therapy

In some embodiments, a bihaptenized vaccine is administered before a cycle of checkpoint inhibitor treatment (treatment cycle) begins. A treatment cycle refers to a period of treatment followed by a period of rest (no treatment) that is repeated on a regular schedule. For example, treatment given for 10 weeks followed by three weeks of rest is one treatment cycle. When this cycle is repeated multiple times on a regular schedule, it makes up a course of treatment. The treatment cycle of the bihaptenized vaccine comprises 1, 2, 3, 4, 5, 6, 7, 8, 9, 10, 11, 12, 13, 14, 15, 16, 17, 18, 19, 20, 21, 22, 23, 24, 25, 26, 27, or 28 weeks of treatment followed by 0, 1, 2, 3, 4, 5, 6, 7, 8, 9, 10, 11, 12, 13, 14, 15, 16, 17, 18, 19, 20, 21, 22, 23, 24, 25, 26, 27, or 28 weeks of rest (non-treatment of the bihaptenized vaccine). The treatment cycle of the checkpoint inhibitor comprises 1, 2, 3, 4, 5, 6, 7, 8, 9, 10, 11, 12, 13, 14, 15, 16, 17, 18, 19, 20, 21, 22, 23, 24, 25, 26, 27, or 28 weeks of treatment followed by 0, 1, 2, 3, 4, 5, 6, 7, 8, 9, 10, 11, 12, 13, 14, 15, 16, 17, 18, 19, 20, 21, 22, 23, 24, 25, 26, 27, or 28 weeks of rest (non-treatment of the checkpoint inhibitor). In some embodiments, both the treatment cycle of the checkpoint inhibitor and the bihaptenized vaccine can be independently repeated 2, 3, 4, 5, 6, 7, 8, 9, 10, or more times. In some embodiments, the treatment cycle of the checkpoint inhibitor and the treatment cycle of bihaptenized vaccine don't overlap. In some embodiments, the treatment cycle of the checkpoint inhibitor and the treatment cycle of bihaptenized vaccine overlap for 1, 2, 3, 4, 5, 6, 7, 8, 9, 10, 11, 12, 13, 14, 15, 16, 17, 18, 19, or 20 weeks. In some embodiments, a bihaptenized vaccine is administered one week before; two weeks before; three weeks before; four weeks before; five weeks before; six weeks before; seven weeks before; eight weeks before; nine weeks before; ten weeks before; eleven weeks before; or twelve weeks before a cycle of checkpoint inhibitor therapy begins. In some embodiments, the first dose of a bihaptenized vaccine is administered and about ten weeks later, a first dose of an immune checkpoint inhibitor is administered, starting the immune checkpoint inhibitor treatment cycle.

In some embodiments, a subject has received prior checkpoint inhibitor therapy before a first administration of a bihaptenized vaccine. In some embodiments, a subject has disease progression following earlier checkpoint inhibitor treatment.

In some embodiments, a bihaptenized vaccine is administered in a series of seven doses, wherein one dose is administered about every seven days during an about seven-week time period. In some embodiments, cyclophosphamide is administered during the treatment cycle of the bihaptenized vaccine. In some embodiments, cyclophosphamide is administered on about day 2, 3, 4, 5, 6, or 7 of the treatment cycle of bihaptenized vaccine. In some embodiments, cyclophosphamide is administered on about day seven of the seven-week of the treatment cycle of bihaptenized vaccine. In some embodiments, cyclophosphamide is administered 1, 2, 3, 4, 5, 6 or 7 days before the second dose of the bihaptenized vaccine. In some embodiments, cyclophosphamide is administered 1, 2, 3, 4, 5, 6 or 7 days after the first dose of the bihaptenized vaccine. In some embodiments, cyclophosphamide is administered 1, 2, 3, 4, 5, 6 or 7 days before the first dose of the bihaptenized vaccine. An exemplary cyclophosphamide dose is about 300 mg/m².

In some embodiments, the bihaptenized vaccine is administered to a subject weekly, bi-weekly, tri-weekly, monthly, bi-monthly, every three months, every four months, every five months, or every six months for treating a cancer. In one embodiment, the subject is a human.

In some embodiments, periodic bihaptenized vaccine booster doses are administered. In some embodiments, a booster is first administered at about 26 weeks following the first bihaptenized vaccine administration. In some embodiments, following a complete first bihaptenized vaccine treatment cycle, vaccine booster doses are administered about every three months. In some embodiments, following a complete first bihaptenized vaccine treatment cycle, vaccine booster doses are administered about every six months. In some embodiments, following a complete first bihaptenized vaccine treatment cycle, vaccine booster doses are administered about once per year.

During the regimen of a bihaptenized tumor vaccine, one or more checkpoint inhibitor therapies are administered to take advantage of the changes in immune signaling. In some embodiments, the patient receives an anti-CTLA-4 agent (e.g. ipilimumab or tremelimumab) and/or an anti-PD-1 agent (e.g. nivolumab, pembrolizumab, or cemiplimab). The immune checkpoint inhibitor can be administered parenterally, such as, in some embodiments, subcutaneously, intratumorally, intravenously. For example, in various embodiments the immune checkpoint inhibitor is administered at a dose of from about 1 mg/kg to about 10 mg/kg intravenously. In various embodiments the immune checkpoint inhibitor is administered at a dose of from about 1 mg/kg to about 5 mg/kg intravenously. The initial dose of the immune checkpoint inhibitor can be administered at least six weeks after the initial bihaptenized vaccine dose, for example in about weeks 6, 7, 8, 9, or 10. In some embodiments, the immunotherapy agent is administered from about 2 to about 6 times (e.g. about 4 times, preferably every three weeks).

In some embodiments, the patient receives a PD-L1 inhibitor, for example, atezolizumab (Tecentriq), Avelumab (Bavencio), and/or Durvalumab (Imfinzi).

In some embodiments, the bihaptenized tumor vaccine is administered intradermally to a metastatic cancer patient previously found to be unresponsive or only partially responsive to immune checkpoint blockade therapy. For example, the bihaptenized tumor vaccine is administered at a dose of from about 8×10⁶ cells to about 22×10⁶ cells in weeks 1, 2, 3, 4, 5, 6, and 7, with ipilimumab administered i.v. at 3 mg/kg. Ipilimumab can be administered every three weeks, beginning in week 10. Alternatively, pembrolizumab can be administered i.v. at 2 mg/kg every three weeks beginning on week 10.

In some embodiments, the bihaptenized tumor vaccine is administered intradermally to a metastatic cancer patient previously found to be unresponsive or only partially responsive to immune checkpoint blockade therapy. For example, the bihaptenized tumor vaccine is administered at a dose of from about 8×10⁶ cells to about 22×10⁶ cells in weeks 1, 2, 3, 4, 5, 6, and 7, with 350 mg of cemiplimab (Libtayo) administered by i.v. infusion over the course of 30 minutes, every three weeks starting from week 10 until disease progression or unacceptable toxicity.

### Pharmaceutical Compositions

In one embodiment, the invention provides a pharmaceutical composition for use in the treatment of cerical cancer as described herein. In some embodiments, the invention provides pharmaceutical compositions, including those described below, for use in the treatment of cervical cancer that is resistant to immune checkpoint inhibitor treatment.

In some embodiments, the invention provides pharmaceutical compositions for treating cervical cancer that has acquired resistance to immune checkpoint inhibitor treatment. In some embodiments, the invention provides pharmaceutical compositions for treating metastatic cervical cancer. In some embodiments, the invention provides pharmaceutical compositions for treating cervical cancer that is resistant to immune checkpoint inhibitor treatment. In some embodiments, the invention provides pharmaceutical compositions for treating cervical cancer that has been previously treated by at least one immune checkpoint inhibitor. In some embodiments, the cervical cancer does not respond to immune checkpoint inhibitor therapy alone.

The pharmaceutical compositions are typically formulated to provide a therapeutically effective amount of a combination as described herein, e.g. a combination comprising at least one immune checkpoint inhibitor and an autologous bihaptenized tumor vaccine. In some embodiments, the at least one immune checkpoint inhibitor is administered before the first dose of an autologous bihaptenized tumor vaccine. In some embodiments, the first dose of an autologous bihaptenized tumor vaccine is administered before the at least one immune checkpoint inhibitor.

Where desired, the pharmaceutical compositions contain a pharmaceutically acceptable salt and/or coordination complex of one or more of the active ingredients. Typically, the pharmaceutical compositions also comprise one or more pharmaceutically acceptable excipients, carriers, including inert solid diluents and fillers, diluents, including sterile aqueous solution and various organic solvents, permeation enhancers, solubilizers and adjuvants.

The pharmaceutical compositions described above are preferably for use in the treatment of cervical cancer as described herein.

In some embodiments, the methods and pharmaceutical compositions of the present invention are for use in the treatment of cervical cancer. In some embodiments, the cervical cancer is squamous cell carcinoma or adenocarcinoma. Squamous cell carcinomas develop from cells in the exocervix. Squamous cell carcinomas most often begin in the transformation zone (where the exocervix joins the endocervix). Adenocarcinomas are cancers that develop from glandular cells. Cervical adenocarcinoma develops from the mucus-producing gland cells of the endocervix. In some embodiments, cervical cancers have features of both squamous cell carcinomas and adenocarcinomas. These are called adenosquamous carcinomas or mixed carcinomas.

The compositions comprising an autologous bihaptenized tumor vaccine may be administered subcutaneously. The compositions comprising an autologous bihaptenized tumor vaccine may be preferentially administered intradermally. In some embodiments the compositions comprising an autologous bihaptenized tumor vaccine is administered intramuscularly. In some embodiments, the compositions comprising an autologous bihaptenized tumor vaccine is administered via intramuscular route into the deltoid or the anterolateral aspect of the thigh. Methods known to those skilled in the clinical arts enable reliable intramuscular injection even with the variation of subcutaneous fat layer thickness and variations between men and women (e.g. Zuckerman, The Importance of Injecting Vaccines into Muscle: Different Patients Need Different Needle Sizes, BMJ 321:1237-1238 (2000).

Described below are non-limiting pharmaceutical compositions and methods for preparing the same.

### Pharmaceutical Compositions for Injection

In preferred embodiments, the invention provides a pharmaceutical composition of an autologous bihaptenized cervical tumor vaccine for injection comprising a vaccine adjuvant.

Other components known to the art may be used in the compositions described herein. Some embodiments of an autologous bihaptenized tumor vaccine may further comprise adjuvants, such as Bacillus Calmette-Guérin (BCG), cytokines (for non-limiting example, granulocyte-macrophage colony-stimulating (GM-CSF)), aluminum gels or aluminum salts, or other adjuvants known to the art to non-specifically stimulate immune response and enhance the efficacy of the immune response to the vaccine. In at least one preferred embodiment, the adjuvant is BCG Tice.

An autologous bihaptenized tumor vaccine may further comprise preservatives known to the art, including without limitation, formaldehyde, antibiotics, monosodium glutamate, 2-phenoxyethanol, phenol, and benzethonium chloride. An autologous bihaptenized tumor vaccine may further comprise sterile water for injection, balanced salt solutions for injections.

### Personalized Diagnostic Kits

The invention also provides kits. The kits include each of (i) one or more single dose filled syringes or vials wherein the syringe or vial fill comprises bihaptenized autologous tumor cells in a pharmaceutically acceptable carrier; (ii) one or more single dose filled syringes or vials wherein the syringe or vial fill comprises a test negative control in a pharmaceutically acceptable carrier; (iii) written instructions; and (iv) a guide for scoring the test results.

Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another active pharmaceutical ingredient. In some embodiments, the another active pharmaceutical ingredient may be separate compositions in separate containers within the kit.

In an embodiment the guide for scoring the personalized diagnostic test results may be a gauge for measuring induration, wheal and flare diameter. In an embodiment the guide may be a series of pictograms, exemplary photos, or diagrams illustrating wheal and flare, induration, or other characteristic features of responses to the diagnostic reagents, for example positive and negative controls. In an embodiment the kit may further comprise a marker for outlining the edges of the responsive skin surface.

In an embodiment the kit may further comprise a unique QR code representing a unique identification code and enabling communication linked uniquely to the personalized diagnostic kit.

Suitable packaging and additional articles for use (e.g. foil wrapping to minimize exposure to air, and the like) are known in the art and may be included in the kit. Kits described herein can be provided, marketed and/or promoted to health providers, including physicians, nurses, pharmacists, formulary officials, and the like.

### Assessing Efficacy

Efficacy of the methods, compounds, and combinations of compounds described herein in treating, preventing and/or managing the indicated diseases or disorders can be tested using various animal models known in the art. Models for determining efficacy of treatments for breast cancer are described in Fantozzi, Breast Cancer Res. 2006, 8, 212. Models for determining efficacy of treatments for ovarian cancer are described in Mullany, Endocrinology 2012, 153, 1585-92; and Fong, J. Ovarian Res. 2009, 2, 12.

Delayed type hypersenditivity (DTH) may be used to monitor efficacy. If DTH is performed to assess anti-tumor immunity, times of DTH testing are: (a) baseline response tested about 14 to about 21 days before the first dose of vaccine; (b) at about vaccine treatment week 10, which is before the first dose of a co-administered checkpoint inhibitor; and (c) at about vaccine treatment week 28, which is after at least two co-administered doses of at least one checkpoint inhibitor. In some embodiments, DTH is performed about every three months after a complete vaccine and checkpoint inhibitor co-administration regimen.

In some embodiments, the invention provides a method of treating a cervical solid tumor with a composition including a combination of an immune checkpoint inhibitor and an autologous bihaptenized vaccine.

The dose of each of the immune checkpoint inhibitor and the autologous bihaptenized vaccine is such that the dose is effective to inhibit signaling between the solid cervical cancer tumor cells and at least one microenvironment selected from the group consisting of macrophages, monocytes, mast cells, helper T cells, cytotoxic T cells, regulatory T cells, natural killer cells, myeloid-derived suppressor cells, regulatory B cells, neutrophils, dendritic cells, and fibroblasts. In some embodiments, the invention provides a method of treating pancreatic cancer, breast cancer, ovarian cancer, melanoma, lung cancer, squamous cell carcinoma including head and neck cancer, and colorectal cancer.

While preferred embodiments of the invention are shown and described herein, such

embodiments are provided by way of example only and are not intended to otherwise limit the scope of the invention. Various alternatives to the described embodiments of the invention may be employed in practicing the invention. Therefore, the spirit and scope of the appended claims should not be limited to the description of the preferred versions contained herein.

The reader's attention is directed to all papers and documents which are filed concurrently with this specification and which are open to public inspection with this specification, and the contents of all such papers and documents incorporated herein by reference. All the features disclosed in the specification (including any accompanying claims, abstract, and drawings) may be replaced by alternative features serving the same, equivalent or similar purpose, unless expressly stated otherwise. Thus, unless expressly stated otherwise, each feature disclosed is one example only of a generic series of equivalent or similar features.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1. Preparing an Autologous Bihaptenized Tumor Vaccine.

### Processing Fresh Tumor Samples

A fresh dissected cervical tumor sample is placed in a sterile, disposable container to which has about 150 ml of Hanks balanced salt solution (HBSS) with 20 μg/ml gentamicin. The tumor sample is stored at 4° C. until further processing. In no case is a sample stored for more than about 96 hours before further processing is initiated.

In a biosafety hood, the cervical tumor tissue is minced and then mechanically dissociated to produce a tumor cell suspension. The tumor cell suspension is counted, aliquotted into sterile cryovials, frozen in a controlled rate freezer, and stored in liquid nitrogen.

### Bihaptenization

The cells are thawed and washed using HBSS. The cells are then irradiated to 2500 cGy total dose of gamma radiation. Next the inactivated cells are divided into two equal aliquots: A first aliquot is modified with DNP by a 30-minute incubation with difluoronitrobenzene (DNFB). A second aliquot is modified with sulfanilic acid (SA) by a 5-minute incubation with the diazonium salt of SA. The haptenized cells are washed with HBSS, counted, and fixed with ethanol at a final concentration of about 37.5% for about 10 minutes.

### Cryopreservation

The haptenized cells from each aliquot are combined, counted, aliquotted, and frozen in a cryopreservation medium comprising about 7% sucrose and about 10% human serum albumin in HBSS. The vaccine aliquots are then frozen and are stored in liquid nitrogen until required for administration.

### Example 2. Personalized Diagnostic Test

The personalized diagnostic test comprises assessing the delayed type hypersensitivity (DTH) reaction to the autologous personalized tumor vaccine and comparing it to a negative control.

The diagnostic is personalized because the reagent eliciting the DTH reaction is an autologous personalized tumor vaccine manufactured using the patient's own tumor tissue.

On the medial surface of a patient's arm or foream, an area of skin is selected for the diagnostic test. This region is termed the test area. A negative control, typically comprising Hank's Balanced Salt Solution and human serum albumen, is intradermally injected to form a small bleb. In an adjacent area, about 3×10⁶ cells of the autologous personalized tumor vaccine are intradermally injected, to form a small bleb.

About 48 hours later, the test area is visually inspected and the diameter of the induration, wheal and flare for the positive control is measured. The diameter of the induration, wheal and flare for the autologous personalized tumor vaccine injection site is measured. A positive response is an induration resulting from the autologous tumor vaccine that is at least 5 mm in diameter.

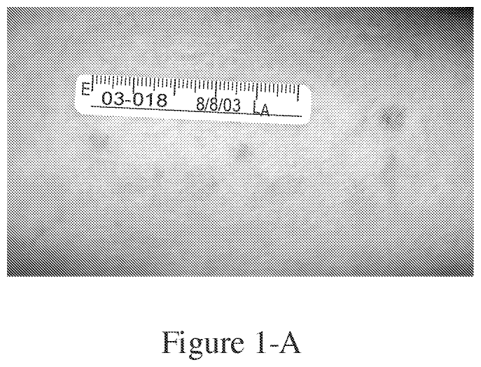

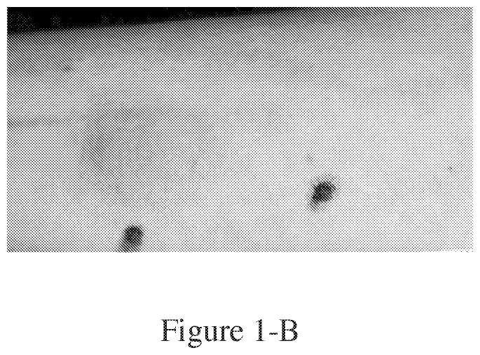

FIG. 1 shows a typical positive, post-vaccine delayed type hypersensitivity response.

The DTH diagnostic response is compared a baseline DTH response performed before the first dose of the vaccine is administered. Typically, this baseline DTH assessment is performed about 14 to 21 days before the first dose of the personalized autologous bihaptenized vaccine is administered. This response is referred to the baseline DTH response.

The DTH response is re-assessed at about 10 weeks after the first vaccine dose and before the first dose of an immune checkpoint inhibitor. The DTH response is re-assessed at about 28 weeks after the first vaccine dose.

### Example 3. Typical Co-Administration Schedule

The table below shows an exemplary administration schedule for bihaptenized vaccine and an immune checkpoint inhibitor, in this example, Keytruda™ (pembrolizumab).

| Event & Timing | Description |
|---|---|
| Week 1, Day 1 | First bihaptenized vaccine administered without |
| | adjuvant |
| Week 1, Day 7 | Cyclophosphamide 300 mg/m² via rapid IV |
| | infusion |
| Week 2, Day 10 | Second bihaptenized vaccine dose administered |
| | with adjuvant |
| Week 3, Day 17 | Third bihaptenized vaccine dose administered |
| | with adjuvant |
| Week 4, Day 24 | Fourth bihaptenized vaccine dose administered |
| | with adjuvant |
| Week 5, Day 31 | Fifth bihaptenized vaccine dose administered |
| | with adjuvant |
| Week 6, Day 38 | Sixth bihaptenized vaccine dose administered |
| | with adjuvant |
| Week 7, day 45 | Seventh bihaptenized vaccine dose administered |
| | with adjuvant |
| Weeks 10, 13, 16, | Keytruda ™ (pembrolizumab) administered |
| 19, 22, and 25 | |
| Week 26 | Eighth bihaptenized vaccine dose administered |
| | with adjuvant; this is a booster dose. |
| Week 28 | Keytruda ™ (pembrolizumab) administered, |
| | continuing according to a typical dosing schedule. |

### Example 4. Combination Therapy Clinical Trial

The clinical trial described compares the efficacy of the combination of a bihapentized tumor vaccine and Yervoy™ (ipilimumab), an immune checkpoint inhibitor. This is a Phase III, randomized, placebo-controlled, double-blind, multi-centered trial in patients with metastatic cervical cancer with measurable metastases. To be eligible for screening, patients have undergone surgery for therapeutic intervention, which yields an adequate amount of cervical tumor cells for preparation of vaccines.

Patients are assigned in a double-blind fashion to Active Vaccine or Placebo Vaccine at a 2:1 ratio (Active Vaccine:Placebo Vaccine). The dose of Active Vaccine is 12±8×10⁶ bihaptenized autologous melanoma tumor cells. The Placebo Vaccine consists of diluent only. An initial dose of Active Vaccine or Placebo Vaccine is administered without BCG followed by low dose cyclophosphamide (300 mg/m² intravenously). Later doses, depending on randomization group, of Active Vaccine or Placebo Vaccine are mixed with Bacillus of Calmette and Guérin (BCG) are administered weekly for at least 6 weeks. At least four doses Yervoy™ are administered to all patients beginning about 3 weeks after the last vaccine dose. This time is at about week 10. Yervoy™ is administered at 10 mg/kg intravenously over 90 minutes every three weeks for four doses, followed by 10 mg/kg every 12 weeks for up to 3 years. In the event of toxicity, Yervoy™ doses are omitted, not delayed.

Patients are evaluated for anti-tumor response by modified RECIST criteria between weeks 24 and 25. At the 6-month point patients who remain on study receive an additional single booster dose of Active Vaccine or Placebo Vaccine mixed with BCG. This booster is followed by doses of Yervoy™ every 12 weeks for up to 3 years. Two additional evaluations for anti-tumor response are done at 9-months and one-year. Then patients are then regularly evaluated for tumor status and adverse events until evidence of progressive disease (PD) potentially requiring additional therapy. Patients without PD remain on the study for up to 5 years.

We claim:

1. A method of treating cervical cancer in a human subject, the method comprising co-administering to the subject in need thereof one or more compositions comprising therapeutically effective amounts of:
(i) at least one immune checkpoint inhibitor; and,
(ii) an autologous bihaptenized vaccine.

2. The method of claim 1, wherein the at least one immune checkpoint inhibitor is administered before the administration of the autologous bihaptenized vaccine.

3. The method of claim 1, wherein the at least one immune checkpoint inhibitor is administered after the administration of the autologous bihaptenized vaccine.

4. The method of any one of claims 1-3, wherein the autologous bihaptenized vaccine is administered every other week for at least eight weeks.

5. The method of any one of claims 1-3, wherein the autologous bihaptenized vaccine is administered once per week for at least six weeks.

6. The method of claim 4 or claim 5, the method further comprising at least one booster injection of the autologous bihaptenized vaccine about six months after the first injection.

7. The method of claim 1, wherein the autologous bihaptenized vaccine is administered every other week until the delayed type hypersensitivity diagnostic test is positive.

8. The method of claim 1, wherein the autologous bihaptenized vaccine is administered weekly until the delayed type hypersensitivity diagnostic test is positive

9. The method of claim 1, wherein the cervical cancer is squamous cell carcinoma or adenocarcinoma.

10. The method of claim 1, wherein the at least one immune checkpoint inhibitor is selected from the group consisting of CTLA-4, PD-1, PD-L1, LAG3, B7-H3, B7-H4, KIR, OX40, IDO-1, IDO-2, CEACAM1, INFR5F4, BTLA, OX4OL, TIM3, and combinations thereof.

11. The method of claim 10, wherein the immune checkpoint inhibitor is PD-1 inhibitor.

12. The method of claim 11, wherein the PD-1 immune checkpoint inhibitor is selected from the group consisting of nivolumab, pembrolizumab, atezolizumab, avelumab, and durvalumab.

13. The method of claim 10, wherein the immune checkpoint inhibitor is CTLA-4 inhibitor.

14. The method of claim 13, wherein the CTLA-4 immune checkpoint inhibitor is selected from the group consisting of ipilimumab and tremelimumab.

15. The method of claim 10, wherein the at least one immune checkpoint inhibitor comprises a PD-1 immune checkpoint inhibitor and a CTLA-4 immune checkpoint inhibitor.

16. A personalized diagnostic test kit, the kit comprising:
(i) one or more single dose filled vial wherein the vial fill comprises bihaptenized autologous tumor cells in a pharmaceutically acceptable carrier;
(ii) one or more single dose filled vials wherein the vial fill comprises a test negative control in a pharmaceutically acceptable carrier;
(iii) written instructions; and
(iv) a guide for scoring the test results.

17. A method of treating cervical cancer, the method comprising:
(i) administering an effective amount of at least one immune checkpoint inhibitor; and,
(ii) administering an effective amount of an autologous bihaptenized vaccine wherein the autologous bihaptenized vaccine is prepared by a process comprising the steps of:
(a) washing a dissected cervical tumor fragment, wherein the fragment is at least about one cm in diameter;
(b) dissociating the cervical tumor fragment into a suspension of cells;
(c) irradiating the suspension of cells with gamma radiation;
(d) dividing the irradiated suspension of cells into at least two approximately equal portions;
(e) haptenizing a first portion with a first haptenization reagent and a second portion with second haptenization reagent;
(f) fixing each portion;
(g) combining the portions; and,
(h) aliquoting cells comprising the product of step (g) into aliquots of about 6×10⁶ to about 50×10⁶ cell/mL.

18. The method of claim 17, wherein the first haptenization reagent is dinitroflurobenzene (DNFB).

19. The method of claim 17, wherein the second haptenization reagent is selected from the group consisting of trinitrochlorobenzene (TNCB), 2,4-difluoronitrobenzene (DNFB), N-iodoacetyl-N′-(5-sulfonic-1-naphthyl)ethylenediamine (AED), sulfanilic acid (SA), trinitrophenol (TNP), 2,4,6-trinitrobenzenesulfonic acid (TNBS) and combinations thereof.

20. The method of claim 17, wherein the dose of the gamma radiation is about 2500 cGy.

21. The method of claim 17, wherein each portion is fixed with about 37.5% ethanol.

22. The method of any one of claims 1 to 15, wherein the cervical cancer is squamous cell carcinoma or adenocarcinoma.
